(19) United States
(12) Patent Application Publication
(10) Pub. No.: US 2018/0239416 A1
(43) Pub. Date: Aug. 23, 2018

(54) VARIABLE IMMERSION VIRTUAL REALITY

(71) Applicant: Bryan Laskin, Wayzata, MN (US)

(72) Inventor: Bryan Laskin, Wayzata, MN (US)

(21) Appl. No.: 15/436,068

(22) Filed: Feb. 17, 2017

(51) Int. Cl. G06F 3/01; G06T 11/60; G06F 3/00; G02C 7/10; G02C 11/00; A61M 16/06; A61M 16/01

(52) U.S. Cl. CPC G06F 3/011; G06T 11/60; G06F 3/002; G06F 3/04842; G02C 11/10; A61M 16/06; A61M 16/01; G02C 7/101

(57) ABSTRACT

An immersion device may include a surround configured for positioning before a user's eyes and having one of an electronic display and a station for receiving an electronic device with an electronic display. The electronic display may be configured for displaying a selected view of environmental footage relating to the surroundings of the user and simultaneously displaying virtual footage unrelated to the surroundings of the user.

### FIELD OF THE INVENTION

The present disclosure relates to systems and methods for providing a virtual experience during dental, medical, orthodontic, chiropractic, or other procedures. More particularly, the present disclosure relates to devices and methods of providing a virtual reality experience during a procedure that may be supplemented by selected exposure to surroundings. Still more particularly, the present disclosure relates to devices and methods of providing varying levels of immersion in a virtual experience during a procedure, such as a dental procedure.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Dental, medical, orthodontic, chiropractic, and other procedures often create angst, anxiety, discomfort, and sometimes pain. Various approaches to comfort patients include selecting soothing music, providing soothing smells, or providing anesthetic such as nitrous oxide or injected or IV drugs, for example. In some cases, less invasive approaches to providing comfort are found to be less effective or even ineffective. Still, more invasive approaches may be overly sufficient and unnecessary or simply not preferred by patients and providers.

### SUMMARY

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to an immersion device having a surround configured for positioning before a user's eyes and having either an electronic display or a station for receiving an electronic device with an electronic display. The electronic display may be configured for displaying a selected view of environmental footage relating to the surroundings of the user and simultaneously displaying virtual footage unrelated to the surroundings of the user. In some embodiments, the electronic display may have a visual input device for capturing environmental footage and displaying a selected view of environmental footage, including redisplaying said environmental footage on the electronic display. In some embodiments, displaying a selected view of environmental footage may include displaying a selected brightness of the environmental footage. Further, displaying a selected view of environmental footage may include displaying a selected clarity of the environmental footage. The surround may include panels in some embodiments, which may be interchangeable. The panels may have a selected amount of opacity. The panels may have an adjustable opacity in some embodiments. The opacity may be adjusted by shutters, an electro-induced tint, or removable films, for example. In some embodiments, the surround may include protective eyewear, which may include lenses and the electronic displaying may be provided on the lenses. In some embodiments, the surround may include a nosepiece configured for supplying gas to the user. Such gas may be nitrous oxide in some embodiments. The surround may additionally include a plurality of external interaction features, such as a button configured for interacting with the electronic display, or a slide or touch screen configured for executing a swipe function on the electronic display. In some features the immersion device may have a toggle feature for toggling between a full immersion experience and no immersion experience or a partial immersion experience.

The present disclosure, in one or more additional embodiments, relates to a method of providing an immersive experience for a user. The method may include adjusting an opacity on one or more panels arranged on a surround, the surround configured for positioning before a user's eyes and having either an electronic display or a station for receiving an electronic device having an electronic display. The method may additionally include arranging the electronic display before the user's eyes. In some embodiments, the surround may include a nose piece, and the method may include arranging the nose piece around the user's nose and adjusting a quantity of gas configured to flow through one or more gas lines coupled to the nose piece.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a perspective view of an immersion system of the present disclosure, as worn by a user, according to one or more embodiments.

FIG. 2 is conceptual view of an immersion system of the present disclosure having multiple levels of opacity, according to one or more embodiments.

FIG. 3 is a perspective view of an immersion system of the present disclosure having a nose piece, according to one or more embodiments.

FIG. 4 is a flow diagram of a method of using an immersion system of the present disclosure, according to one or more embodiments.

### DETAILED DESCRIPTION

The present application, in one or more embodiments, relates to an immersive experience system such as a virtual and/or augmented reality system or the like that may be used during dentistry procedures or other procedures and may provide a mental distraction to a patient and/or act as a drugless anesthetic. The system may be dual or multi-purpose and, for example, may also act as protective eyewear and/or may include a combination of a gas nose piece, for example. The system may include varying levels of immersion with attachable panels that can be selected to close out more or less of the surrounding environment. The system may also provide varying levels of immersion by allowing for adjustment of the physical and/or digital opacity of the system such that a user may choose to focus on the physically surrounding environment or the virtual experience or a combination of both. The system may include features for interfacing with a smartphone or other device including exterior push buttons for actuating corresponding buttons on the phone or for actuating screen taps or touches and may also include sliding or swiping mechanisms that may allow for swipe gestures to be executed and recognized by the smartphone.

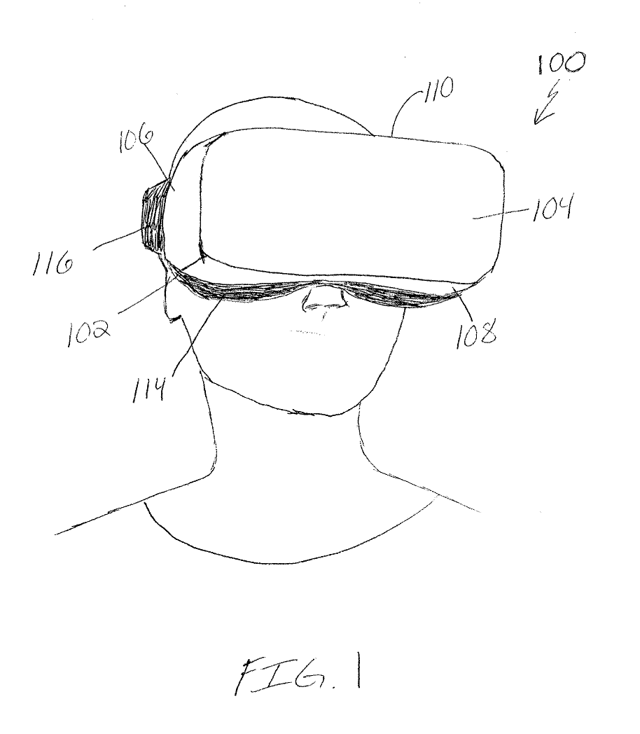

Turning for example to FIG. 1, in one or more embodiments, an immersion system 100 may generally include a surround 102 and an electronic display 104. The surround 102 may be configured for positioning before or in front of a user's eyes or face. In some embodiments, the surround 102 may further be configured to position the electronic display 104. The electronic display 104 may be configured to present images, videos, interactive media, and/or other media to the user to distract the user from a procedure or other event. In one or more embodiments, the surround 102 and/or the electronic display 104 may provide a desired or selected amount of opacity relative to the surrounding environment.

The surround 102 may generally be arranged over or in front of a user's eyes, so as to position the electronic display 104 in the user's view. In some embodiments, the surround may include a frame configured to support the electronic display 104. Additionally or alternatively, the surround 102 may include one or more panels. For example, the surround 102 may have one or more side panels 106, a bottom panel 108, and/or a top panel 110. In some embodiments, one or more panels may be combined in a single panel. For example, in some embodiments, the bottom panel 108 may wrap around the sides of the surround 102 and extend upward to provide side panels as well. In some embodiments, the panels may encompass a single component extending around the top, bottom, and sides of the surround 102. The panels may generally be configured to block or reduce the user's view of the surrounding environment. In some embodiments, the panels may be configured to form an enclosure with the user's face or against the user's face. The panels may help to direct the user's attention to the electronic display 104 in some embodiments. Additionally, in some embodiments, the panels may provide protection for the user's eyes. In one or more embodiments, the surround 102 may include or provide additional protective eyewear and/or a nose piece.

In some embodiments, the surround 102 may be generally contoured to fit comfortably and/or securely on a user's face. Similarly, the surround 102 may be contoured to wrap around a user's face so as to enclose the user's view area. For example, the side panels 106, bottom panel 108, and/or top panel 110 may each have various appropriate contouring. The bottom panel 108, for example, may have a ridge or opening configured to fit around the user's nose and/or mouth. As another example, the top panel 110, may have a curved shape configured to wrap around a user's brow, above the user's eyes. The panels 106, 108, 110 and/or other components of the surround 102 may be constructed of glass, one or more plastics, paper, and/or any other suitable components.

In some embodiments, the surround 102 may include padding 114 or cushioning, such as a foam padding, arranged between one or more panels and the user's face. The padding 114 may be configured to provide a comfortable and/or tight fit. In some embodiments, the padding 114 may be configured to help enclose the user's view.

Additionally, in some embodiments, the surround 102 may have a head strap 116 coupled to a side panel 106 at each of two ends, for example. The head strap 116 may be or include an elastic component, one or more clips, rings, or other adjustable components configured to help provide a comfortable and/or secure fit of the surround 102 on a user's head. In other embodiments, the surround 102 may have one or more arms or temples configured to be placed behind a user's ears, such as those typically arranged on eyeglasses or safety goggles. In some embodiments, surround 102 may be provided in more than one size.

The electronic display 104 may be arranged on, in, or incorporated into the surround 102. For example, in some embodiments, the electronic display 104 may be provided by a smartphone or other electronic display positioned or stationed in the surround 102, such that the smartphone's screen may provide the electronic display 104. For example, the surround 102 may have an opening or slot for receiving a smartphone or other device. In other embodiments, the electronic display 104 may include one or more screens or displays incorporated into the surround 102. The electronic display 104 may include, for example, a projected image or a full area display such as a LED, LCD, plasma, or other full area display. The electronic display 104 may be relatively flat, curved, or alternately shaped. In some embodiments, one or more panels 106, 108, 110 may have or include a screen or a portion of a screen, such that the electronic display 104 wraps around the user's field of vision. In one or more embodiments, external interaction features may be provided to interact with the electronic display.

The electronic display 104 may be configured to provide a virtual experience, similar to a virtual reality and/or augmented reality experience, in some embodiments. For example, in a virtual reality experience, the electronic display 104 may present one or more videos and/or images to the user. In some embodiments, the electronic display 104 may incorporate a camera to provide images or footage of a surrounding environment together with, or as part of, the virtual experience. For example, the environmental images or footage may be permitted to bleed through virtual images or footage. In other embodiments, the virtual experience may be overlaid on or integrated with images or footage of the surrounding environment. In some embodiments, where for example the electronic display 104 includes a smartphone, the smartphone's camera may be used to provide on the smartphone's screen images of the environment in which the user is positioned. In the context of dental work, for example, the smartphone's camera may be used to provide the user with a current view or footage of the exam room. In some embodiments, the immersion system 100 may then provide a virtual experience by displaying images overlaid or integrated with the environment images. In some embodiments, a user may have the option of switching or toggling between a current, un-altered view of the environment and a virtual experience display. That is, where a user experiences motion sickness or has a desire to view something or someone in the room, the user may view the current environment without removing the immersion system 100. In other embodiments, the user may have the option of increasing an amount or strength of environment footage or images that may be viewable with the virtual footage or images.

Additionally or alternatively, in some embodiments, the electronic display 104 may be configured to provide a view or footage of the surrounding environment adjacent to virtual footage or images. That is, the electronic display 104 may be divided into two portions, with a first portion dedicated to displaying environmental footage and a second portion dedicated to displaying virtual footage. In some embodiments, the display may be divided equally, such that the two portions are of equal size. The two portions may be arranged on either side of a dividing horizontal or vertical line, for example. In other embodiments, the first and second portions may be of different sizes, where one portion is dominant to the other. For example, the first portion may be provided as a small box or screen embedded in a corner of the larger second portion. In such embodiments, a user may have the option to toggle the two portions, switching the dominant portion between the first and second portions. In some embodiments, the two portions may be arranged such that the first portion is arranged before, an generally viewable with, one of the user's eyes, while the second portion is arranged before, or generally viewable with, the other of the user's eyes. In still other embodiments, the electronic display 104 may be divided into portions in different ways.

In some embodiments, one or more portions of the immersion system 100 may be configured to be disposable and/or recyclable. For example, portions of the immersion system 100 may be constructed of relatively inexpensive components in some embodiments. In some embodiments, disposable components may allow users to easily have a sanitized or sterile system. For example, the surround 102, or portions thereof may be configured to be disposable and/or recyclable in some embodiments. In such embodiments, the electronic display 104 may be reusable, or may be, for example, provided by a smartphone or other electronic device. In other embodiments, the electronic display 104 may be configured to be disposable and/or recyclable.

### Removable Panels

In some embodiments, the immersion system 100 may include one or more removable panels. For example, the one or more panels 106, 108, 110 of the surround 102 may be removable and/or attachable. The removability of the panels may allow for closing off more or less of the space surrounding the electronic display 104 and, as such, allowing a user to select a level of immersion based on how enclosed the immersion system 100 is. A user may have the option, for example, of wearing the immersion system 100 without any panels, with limited panels, or with all panels, in order to customize the experience. For example, and with particular relevance to dentistry, a user may wish to attach the bottom panel 108 configured for positioning below the electronic display 104, but above a user's mouth, so as to close off and/or block the view of the dental tools and/or the procedure from the user's eyes. Top panel 110 and side panels 106 may additionally help to close off and/or block views of the dental or other environment. In one or more embodiments, the panels may be pivotable to open and/or close rather than, or in addition to, attaching/removing them.

The panels 106, 108, 110 may be attachable to the electronic display 104 and/or attachable to one another where the panels extend around the display and are positioned adjacent to one another, for example. The panels may be attachable via one or more of several available types of attachment mechanisms. In some embodiments, the panels may include an elongated shaft or rib along an edge that may be receivable by an elongated jaw or channel on an adjacent panel or on the display, for example. The shaft or rib of the panel may be forcibly inserted into the jaw or channel causing the jaw or channel to open slightly and allow the shaft or rib to pass into the mouth of the jaw or channel. The jaw or channel may then resiliently close down on an opposing side of the shaft or rib to maintain the shaft or rib in place. In other embodiments hooks and dowels, loops, or slots may be used such that a panel may be attached or removed by sliding a hook along an adjacent panel to engage or disengage a dowel, loop, or slot. In other embodiments, flexible hook and loop tabs, such as Velcro®, may be folded into an overlapping condition with an adjacent panel or display to secure the panel. Still other types of attachment mechanisms may be used.

### Opacity

In any of the embodiments described, a particular amount of opacity may be provided to allow the user to perceive the surrounding environment as much as desired. For example, some users may be comfortable and/or or may wish to be completely visually isolated from their environment and may, thus, prefer a fully opaque device allowing for full immersion into a virtual experience. Other users may not feel comfortable with full immersion due to a lack of connection with their surroundings, motion sickness issues, or other concerns. In these situations, the user may prefer to have some visual sense of their environment and may appreciate the opportunity to select and/or adjust the level of immersion based on selecting and/or adjusting the amount of visual sense they have of the surrounding environment. In some embodiments, the opacity may be a physical opacity provided by the material or the opacity may be a virtual or digital opacity provided by a display, for example. The opacity may be set or adjusted to any level ranging from 0% to 100% in some embodiments.

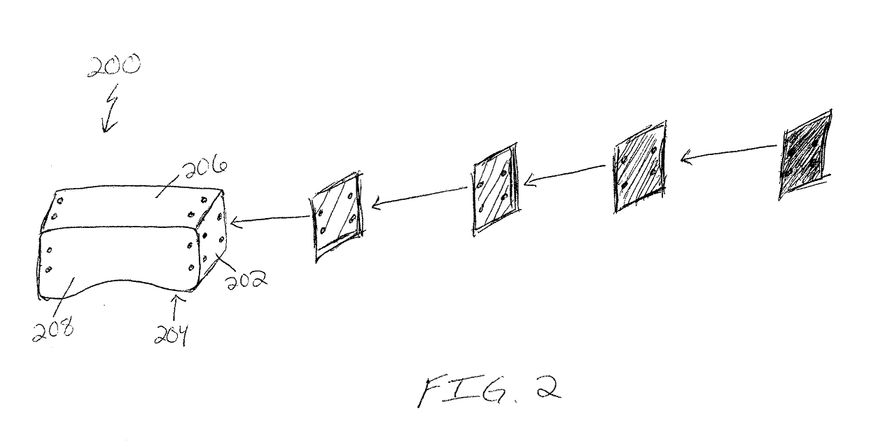

Turning to FIG. 2, for example, an immersion system 200 of the present disclosure may have one or more side panels 202 a bottom panel 204, and/or a top panel 206 arranged around an electronic display. Additionally, in some embodiments, a front panel 208 may be arranged outside of the display. Each of the panels 202, 204, 206, 208 may have an opacity that may be adjusted physically or electronically. It may be appreciated that although the user's physical view of the environment may be blocked by the display, where a camera is used to provide a display of the native environment, the front panel 208 may provide an opacity with respect to the camera's view of the environment. As shown in FIG. 2, opacity on each panel may range between substantially or entirely clear and substantially or entirely opaque.

With respect to the physical opacity, various levels of opacity may be provided by providing a particular tint on one or more portions of the device or system. For example, one or more panels 202, 204, 206, 208 may have varying levels of tint. Tint may be provided by way of films or members having a particular material opacity or a surface treatment such as a tint film, printed pixels, printed lines, or other approaches to providing a tint or dimness may be provided. In one or more embodiments, one or more panels may be photochromatic panels configured to darken automatically in response to varying light intensities.

In one or more embodiments, this physical opacity may be adjusted to the user's preference. For example, as described above, panels of the system may be removable, and thus may be swapped out with other panels to provide a desired opacity. That is, several panels having varying opacities may be provided for a particular location on the device allowing a user to choose an opacity and swap out one panel for another based on their preference. In other embodiments, the tint may be adjustable in-situ by way of chemical tints, films, electro-induced tints, adjustable shutters and the like. In some embodiments, the physical opacity may be used on features surrounding the display. In other embodiments, for example, where the display is a projected display, the physical opacity may be used on the panel or panels on which a display is provided. The adjustable opacity may, thus, allow a user to perceive a desired amount of his/her surroundings while simultaneously experiencing virtual footage.

With respect to a virtual or digital opacity, such may be provided by capturing footage or imagery of the surrounding environment and redisplaying such imagery or footage on the display. For example, the digital display device may be in the form of a smartphone or other electronic device that has a camera or other visual input device for capturing imagery and or footage of the surrounding environment. Such imagery or footage may then be redisplayed on the display to cause the environment to bleed through onto the display (i.e., to make the electronic display appear transparent or semi-transparent). Depending on the user's preference, the amount of bleed through environment displayed may create a display that is fully opaque (i.e., no environmental bleed through) or a selected amount of opacity (i.e., some selected amount of environmental bleed through).

The amount of clarity of such footage or imagery may be adjusted digitally by increasing or decreasing the brightness of the environmental footage relative to the virtual footage or by adjusting the pixels or lines (i.e. vertical, horizontal, or diagonal lines) that are dedicated to showing the environmental footage. Still other approaches to adjusting the clarity of the environmental footage relative to the virtual footage may be provided.

In one or more embodiments, the display may have a perspective adjustment such that the perspective of the environment aligns well with the actual feature of the environment viewable through items having a selected or adjustable physically opacity. For example, depending on the user and the fit of the electronic display relative to the user's face, the electronically displayed environment may or may not align well with the view of the surrounding actual environment. In one or more embodiments, the perspective adjustment may allow for physically or digitally adjusting the distance of the display or focal point from the user's eyes. In one or more additional or alternative embodiments, the perspective adjustment may allow for digitally stretching or digitally shrinking the electronic display in a vertical or a horizontal direction to allow for alignment of features in the display with the view of surrounding features. Still other mechanisms and approaches to providing a perspective adjustment may be provided.

The adjustable opacity, whether physical or digital, may allow a user to perceive his/her surroundings while also perceiving the imagery on the display and the user may consciously or unconsciously choose to focus on one or the other or a combination of both. The adjustability of the opacity may allow a user to affect the virtual footage by allowing a lot of the environment to come through the device or by reducing the amount of environment that comes through. Accordingly, a user may be provided with the option to reduce the quality or clarity of the virtual footage by increasing the exposure to the environment. While reducing the quality or clarity of the virtual footage may seem undesirable, some users may prefer to have a better sense of their surroundings and may elect to sacrifice quality or clarity of virtual footage for a better sense of their surroundings.

In one or more embodiments, room lighting may be adjusted to further accommodate and/or affect the perception of the user. For example, brighter room lighting may allow a user to better perceive his/her surroundings. However, depending on the source of the light, it may also create glare or other reflections and hamper the user experience of the virtual footage. Accordingly, room lighting may be yet another feature allowing a user to select a balance between exposure to virtual footage and exposure to his/her surroundings.

In still other embodiments, a toggle may be provided to toggle between environmental footage and virtual footage. That is, for example, where a user prefers full immersion or prefers to have the virtual footage be uninterrupted by environmental footage or distraction, a toggle may be provided to toggle back and forth between the virtual footage and environmental footage. In one or more embodiments, the toggle may be in the form of a button or other interaction feature such as the ones discussed below. Moreover, actuating the toggle may affect the display, the adjustable opacity of non-display components, or both. For example, if the device is in full immersion mode, the display may be displaying solely virtual footage and the surrounding panels or other aspects of the device may be substantially or fully opaque. Upon actuating the toggle, the display may transition from displaying solely virtual footage to displaying solely environmental footage. Moreover, the surrounding panels or other aspects of the device may transition from being substantially or fully opaque to being substantially or fully transparent. In some embodiments, the toggle may be used to toggle between full immersion and partial immersion rather than full immersion and no immersion. Still other ranges of toggling may be provided.

### Nose Piece

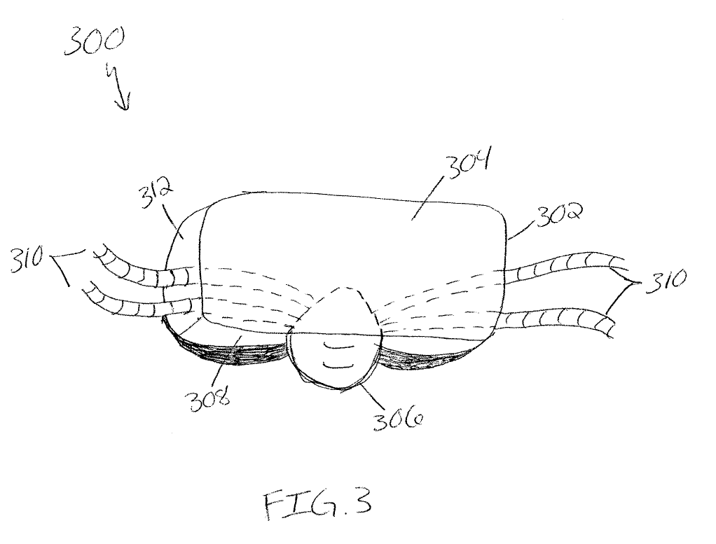

In one or more embodiments, an immersion system of the present disclosure may incorporate, or be incorporated into, a nose piece, such as those used to deliver gases, mixtures of gases, or other fluids to the respiratory system of a patient. For example, turning to FIG. 3, an immersion system 300 is shown. The immersion system 300 may generally have a surround 302, a display 304, and a nose piece 306. In one or more embodiments, the nose piece 306 may extend from or couple to a bottom panel 308 of the surround 302. That is, the nose piece 306 may extend from a bottom side or edge of the bottom panel 308 and may extend across and over the nose of the user. The nose piece 306 may be sized and shaped to nestle over the nose of a user similar to the way known nose pieces do, such that gas from the nose piece is prevented or interrupted from leaking up and into a user's eyes. In some embodiments, the nose piece 306 may be configured to be arranged over a user's nose without substantially interfering with a procedure being performed on the user's mouth. In other embodiments, the nose piece 306 may be configured to also be arranged over the user's mouth, for example, for other procedures or purposes outside of dental work. The nose piece 306 may be permanently attached to the immersion system 300, or the nose piece may be selectively detachable such that the system may be used with or without the nose piece, depending on the procedure being performed. In one or more embodiments, a series of interchangeable nose pieces may be provided to accommodate different sizing requirements of the user and/or for disposal of used nose pieces and replacement with new nose pieces, for example. In one or more embodiments, the entire immersion system 300 with the nose piece 306 may be disposable.

In some embodiments, one or more gas lines 310 may be connected to the nose piece 306 allowing for the delivery of nitrous oxide, oxygen, and/or other gases to the user. As shown in FIG. 3, in some embodiments, the one or more gas lines 310 may be arranged through a side panel 312 of the surround 302 to reach the nose piece 306. That is, the one or more gas lines 310 may be configured to run beneath or behind the surround 302 and/or display 304 in order to connect to the nose piece 306 in some embodiments. In other embodiments, the one or more gas lines 310 may run outside of the surround 302 and display 304.

In one or more embodiments, the nose piece 306 may be pivotable relative to the surround 302 and/or display 304, such that the user or the provider may selectively engage the nose piece without interrupting the virtual experience. For example, the nose piece 306 may be connected to the surround 302 and/or display 304 with a hinge, magnet, or other easily manipulable feature allowing the nose piece to be adjusted, removed, pivoted, or otherwise manipulated without substantially affecting the immersion system or device.

### Protective Eyewear

An immersion system of the present disclosure may be multi-purpose and may function as both an immersive experience device and a protective eyewear device. For example, the system may include modified protective eyewear such as modified protective eye glasses or modified protective eye goggles, for example. In this embodiment, the protective eye glasses, eye goggles, or other eyewear may include an electronic display in the location of the lenses and/or in other locations on the eyewear. In one or more embodiments, the eyewear may include a docking feature allowing for placement of a smartphone or other electronic device that may function as an electronic display. As such, the system may include modified protective eyewear that also provides an immersive experience such as by having video functionality in the lenses or other aspects of the protective eyewear.

In one or more embodiments, where the protective eyewear includes side shields and/or top or bottom shields, such surfaces or location may also include an electronic display.

While the dual purpose concept of an immersive experience together with protective eyewear has been discussed in the context of modified protective eye glasses or goggles, alternatively, the system may include modified virtual reality systems rather than modified eyewear. For example, a virtual reality headset may be modified to include eyeglass stems for ease of use such as to allow a user with an easy on or easy off option for using the system. In other embodiments, the virtual reality headset may be mounted to a procedure chair and provided with a swing arm or other pivot mechanism allowing the headset to be moved into position and placed before and/or on the user's face without attaching it to the user's head, for example. In either case, the system may provide an immersive experience and may further create a protective zone around a user's eyes to establish protective eyewear.

### Interaction Features

In one or more embodiments, an immersion system of the present disclosure may include external interaction features for interacting with and/or controlling the electronic display. In one or more embodiments, the external interaction features may be configured for interacting with and/or controlling a smartphone or other electronic device forming the display. The external interaction features may include one or more buttons, slides, touch screens, switches, levers, or knobs, for example. The external interaction features may be particularly adapted for adjusting a particular aspect of the display or the function of the external interaction features may be controlled by the device they are physically, electrically, or wirelessly in communication with.

In one or more embodiments, the external interaction features may be physically in communication with the display or an electronic device having a display. The external interaction features may, thus, be pushed, slid, switched, adjusted, rotated, or otherwise manipulated to control a particular aspect of the display. In some embodiments, for example, the external interaction features may be positioned and arranged to particularly align with corresponding buttons or features on a smartphone or electronic device similar to the way protective covers align. In other embodiments, the manipulable arms, levers, or rods may be in physical communication with the external interaction features and may be placed in physical communication with corresponding features on the electronic display or smartphone or electronic device. In some embodiments, external interaction features may be positioned and arranged to particularly align with a screen of a smartphone or electronic device, allowing a user to operate one or more touch screen features of the smartphone or electronic device using the external features. For example, one external interaction feature may allow a user to perform a slide or swipe function on the screen or display of the smartphone or device.

In other embodiments, the external interaction features may be in electrical communication with the display or an electronic device having a display. In one or more embodiments, the immersion system may have its own power source and in other embodiments, the immersion system may plug into a smartphone or other electronic device to receive power. In either case, the external interaction features may be electrically powered by the power source and manipulation of the features may send or interrupt electronic signals to the display or electronic device providing the display.

In still other embodiments, the external interaction features may be in wireless communication with the display or an electronic device having a display. Such wireless communication may be a short range communication such as that provided by a Bluetooth connection, for example. Still other communication technologies may be used such as Zigbee or other technologies.

In one or more embodiments, the various manipulations of the external interaction features may be particularly adapted to accommodate interactions with a smartphone, for example. That is, depressing buttons on the immersion system or touching a touch screen may be akin to pressing buttons such as power, volume, the home button, or other common button type features on a phone. In one or more embodiments, a slide feature or a swipe on a touch screen on the immersion system may be configured to actuate swipe features on the smartphone, for example. Still other interactions with a smartphone may be provided such as by using switches, levers, or knobs on the immersion system.

### Method of Use

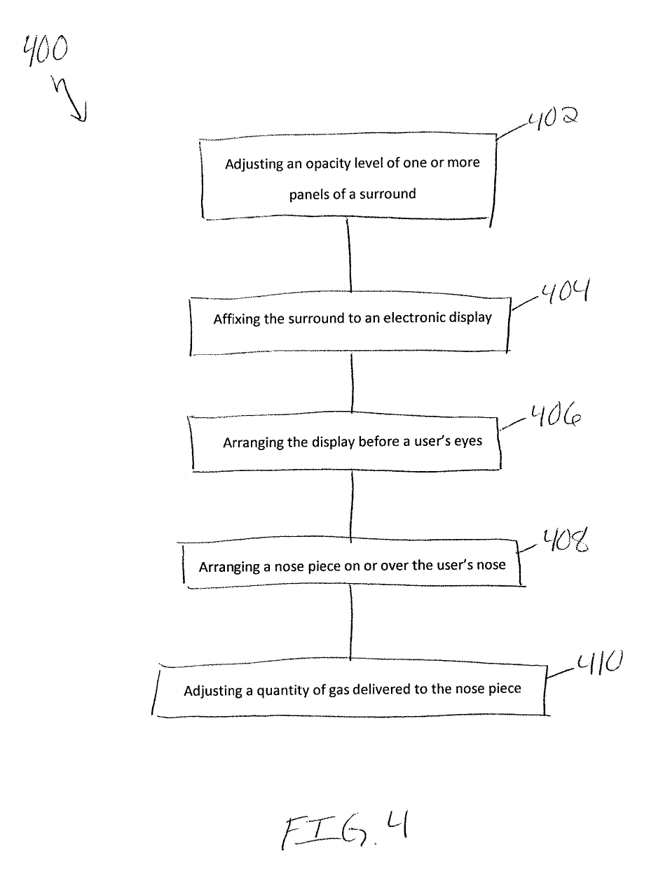

Turning now to FIG. 4, a method 400 of using an immersion device of the present disclosure is shown. The method 400 may include adjusting an opacity level on one or more panels of a surround 402, affixing the surround to an electronic display 404, arranging the display before a user's eyes 406, arranging a nose piece on or over the user's nose 408, and adjusting a quantity of gas delivered to the nose piece 410.

Adjusting an opacity level on one or more panels of the surround 402 may be accomplished by various physical and/or electronic means. In some embodiments, the opacity may be adjusted by physically attaching one or more removable or attachable panels in some embodiments. In other embodiments, the opacity may be adjusted by arranging a tint or other physical component over one or more panels. In still other embodiments, the one or more panels may be adjusted electronically, as described above. In some embodiments, the opacity level may be adjusted by a dentist or dental assistant, for example. In other embodiments, the opacity level may be adjusted by the user.

In some embodiments, the method 400 may additionally include affixing the surround to an electronic display 404. The surround may couple to the display by any suitable attachment mechanism. In other embodiments, the electronic display may be arranged in a slot or opening in the surround configured to receive the display. For example, where the display is provided by a smartphone or other electronic device, the smartphone or device may be arranged in an slot or opening in the surround, or may be arranged on, in, or with the surround in any other suitable way.

Arranging the display before a user's eyes 406 may include, for example, arranging one or more head straps of the surround around a user's head. In other embodiments, arranging the display before a user's eyes may include arranging one or more temples or arms over and/or behind the user's ears. In other embodiments, arranging the display before the user's eyes may include pivoting or swinging an arm or device coupled to the display, so that the display is brought before the user's eyes.

Arranging a nose piece on or over the user's nose 408 may include attaching the nose piece to the surround and/or display in some embodiments. In other embodiments, arranging the nose piece may include pivoting the nose piece to bring it over the user's nose.

Adjusting a quantity of gas delivered to the nose piece 410 may include adjusting a quantity, concentration, or flow rate of nitrous oxide, oxygen, and/or other gases that may be delivered to a user through the nose piece. As described above, the one or more gasses may be delivered via one or more hoses or gas lines.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free” of or “generally free of” an ingredient or element may still actually contain such item as long as there is generally no measurable effect thereof

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

1. An immersion device, comprising:
a surround configured for positioning before a user's eyes and having one of an electronic display and a station for receiving an electronic device with an electronic display, wherein the electronic display is configured for displaying a selected view of environmental footage relating to the surroundings of the user and simultaneously displaying virtual footage unrelated to the surroundings of the user, and a nosepiece, said nosepiece connected to a plurality of gas lines, said disposed within the surround for delivering a gas.

2. The immersion device of claim 1, wherein the electronic display comprises a visual input device for capturing environmental footage and displaying a selected view of environmental footage comprises redisplaying said environmental footage on the electronic display.

3. The immersion device of claim 2, wherein displaying a selected view of environmental footage comprises displaying a selected brightness of said environmental footage.

4. The immersion device of claim 3, wherein displaying a selected view of environmental footage comprises displaying a selected clarity of said environmental footage.

5. The immersion device of claim 1, wherein the surround comprises panels.

6. The immersion device of claim 5, wherein the panels are interchangeable.

7. The immersion device of claim 5, wherein the panels have a selected amount of opacity.

8. The immersion device of claim 5, wherein the panels have an adjustable opacity.

9. The immersion device of claim 8, wherein the surround comprises protective eyewear.

10. The immersion device of claim 9, wherein the protective eyewear comprises lenses and the electronic display is provided on the lenses.

11-12. canceled

13. The immersion device of claim 12, wherein the gas is nitrous oxide.

14. The immersion device of claim 1, wherein the surround comprises a plurality of external interaction features.

15. The immersion device of claim 14, wherein the plurality of external interaction features comprises a button configured for interacting with the electronic display.

16. The immersion device of claim 14, wherein the plurality of external interaction features comprises a slide or touch screen configured for executing a swipe function on the electronic display.

17. The immersion device of claim 1, further comprising a toggle feature for toggling between a full immersion experience and no immersion experience or a partial immersion experience.

18. The immersion device of claim 8, wherein the adjustable opacity is provided with shutters, an electro-induced tint, or removable films.

19. A method of providing an immersive experience for a user, the method comprising:
adjusting an opacity on one or more panels arranged on a surround, the surround configured for positioning before a user's eyes and having one of an electronic display and a station for receiving an electronic device having an electronic display;
arranging the electronic display before the user's eyes;
and wherein the surround comprises a nose piece configured for arranging around a user's nose, the method further comprising:
arranging the nose piece around the user's nose; and
adjusting a quantity of gas configured to flow through one or more gas lines disposed within the surround to the nose piece.

20. canceled
